(19) United States
(12) Patent Application Publication
(10) Pub. No.: US 2021/0077376 A1
(43) Pub. Date: Mar. 18, 2021

(54) TOPICAL COMPOSITION

(71) Applicant: DSM IP ASSETS B.V., Heerlen (NL)

(72) Inventor: Christine MENDROK-EDINGER, Kaiseraugst (CH)

(21) Appl. No.: 16/954,027

(22) PCT Filed: Dec. 17, 2018
(86) PCT No.: PCT/EP2018/085132

§ 371 Date: Jun. 15, 2020

(30) Foreign Application Priority Data
Dec. 19, 2017 (EP) 17208373.5

(51) Int. Cl. A61K 8/89; A61K 8/06; A61K 8/25; A61K 8/35; A61K 8/42; A61K 8/55; A61K 8/97; A61Q 17/04

(52) U.S. Cl. CPC A61K 8/89; A61K 8/062; A61K 8/25; A61K 8/35; A61K 2800/412; A61K 8/55; A61K 8/97; A61Q 17/04; A61K 8/42

(57) ABSTRACT

The present invention relates to topical compositions comprising a polysiloxane-based UV filter, hydroxyacetophenone and panthenol, as well as their use in sunscreens.

The present invention relates to topical compositions comprising a polysiloxane-based UV filter, hydroxyacetophenone and panthenol, as well as their use in sunscreens.

Sunscreens should be effective and safe. In this respect sunscreens contain different UVB and UVA filter. Some of them are lipophilic liquid substances which lead to a greasy residue on skin. Other UV filter are solid and need to be dissolved in emollients, also leading to a greasy residue on skin. The greasy residue on skin leads to a stronger stickiness of sand, an adverse effect consumers suffer from at the beach. To prevent this, consumers tend to use less sunscreen but at the same time they are less protected. The adverse effects of UV radiation on skin are well known. It is therefore important for the industry to provide solutions for such kind of issues to guarantee, that consumers apply sufficient sunscreen for appropriate protection.

Moreover, today's sun care products need skin-friendly preservatives or preservative boosters, which can be an alternative to conventional preservatives. Another need of sun care products is to improve moisturization and prevent or reduce irritations on the skin.

It was therefore the object of the present invention to remedy the disadvantages of the prior art and to develop sun care products comprising one or more UV filters, a mild preservative and a soothing agent which after application exhibit a reduced stickiness resulting in a reduced affinity of sand to the skin.

Surprisingly, it has been found that compositions comprising a specific soothing agent and a specific preservative in combination with a polysiloxane-based UV filter exhibit a reduced stickiness resulting in a reduced affinity of sand to the skin.

Thus, the invention relates in one aspect to topical compositions comprising a polysiloxane-based UV filter, a hydroxyacetophenone and panthenol.

The term “topical” is understood here to mean external application to keratinous substances, which are in particular the skin, scalp, eyelashes, eyebrows, nails, mucous membranes and hair, preferably the skin.

Panthenol (INCI) is also referred to as D-panthenol, dexpanthenol, provitamin B5, or (+)-(R)-2,4-Dihydroxy-N-(3-hydroxypropyl)-3,3-dimethylbutyramid. Panthenol improves hydration, reduces itching and inflammation of the skin, improves skin elasticity, and accelerates epidermal wound healing. Panthenol is e.g. commercially as D-Panthenol at DSM Nutritional Products Europe Ltd.

The term hydroxyacetophenone refers to o-, m- or p-hydroxyacetophenone. Particularly preferred in all embodiments of the present invention is p-hydroxyacetophenone [CAS 99-93-4] which is also called 1-(4-hydroxyphenyl)-ethanone and which is e.g. commercially available at Symrise as SymSave® H.

According to a further embodiment, the polysiloxane-based UV filter in the topical composition has a chromophore residue of the benzalmalonate type.

According to a further embodiment, the polysiloxane-based UV filter in the topical composition is a compound according to formula Ia or Ib:

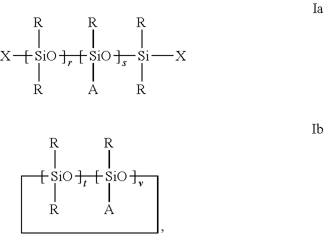

wherein

X is R or A;

A is selected from formula IIa, IIb or IIc:

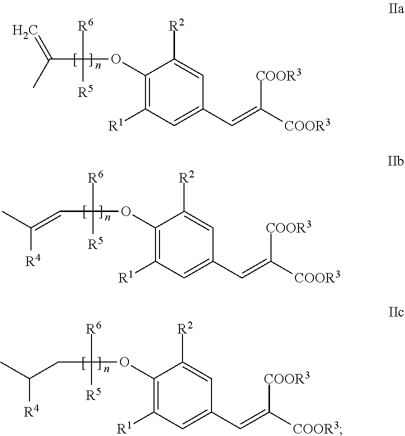

R is hydrogen, C₁₋₆-alkyl or phenyl;

R¹ and R² are each independently hydrogen, hydroxy, C₁₋₆-alkyl or C₁₋₆-alkoxy;

R³ is C₁₋₆-alkyl;

R⁴ is hydrogen or C₁₋₆-alkyl;

R⁵ and R⁶ are each independently hydrogen or C₁₋₆-alkyl;

r is from 0 to 250;

s is from 0 to 20;

r+s is at least 3;

t is from 0 to 10;

v is from 0 to 10;

v+t is at least 3; and

n is from 1 to 6;

with the proviso that when s is 0, at least one X is A.

The term “C₁₋₆-alkyl” refers to groups such as methyl, ethyl, propyl, isopropyl, butyl, sec. butyl, isobutyl, pentyl and neopentyl. The term “C₁₋₆-alkoxy” refers to the corresponding alkoxy groups.

In all embodiments of the present invention, R is preferably methyl.

The residues R¹ and R² are preferably hydrogen, methoxy or ethoxy, more preferably hydrogen, or one of R¹ and R² is hydrogen and the other is methyl, methoxy or ethoxy.

The residues R³ are preferably methyl or ethyl, more preferably ethyl.

Preferably, R⁴ is hydrogen or methyl, R⁵ and R⁶ are hydrogen and n is 1.

The polysiloxane compounds having a group A of the general formula IIa and IIb and their preparation are described in European patent application EP-A0538431. These polysiloxane compounds are most preferred.

The polysiloxane compounds having a group A of the general formula IIc and their preparation are described in the European patent application EP-A 0358 584.

In the linear polysiloxane compounds according to formula Ia the chromophore carrying residue A may be connected to the end groups of the polysiloxane (X=A) or may be statistically distributed over the polymer.

Linear polysiloxane compounds wherein the chromophore carrying residue A is statistically distributed are preferred. Said preferred polysiloxane compounds have at least one unit carrying the chromophore residue (s=1), preferably s has a value of from about 2 to about 10, more preferably a statistical mean of about 4. The number of r of the other silicone units present in the polysiloxane compounds is preferably about 5 to about 150, more preferably a statistical mean of about 60.

Polysiloxane compounds wherein 20% or less, preferably less than 10%, of the total siloxane units are units carrying a chromophore residue are preferred with respect to cosmetic properties.

The ratio of polysiloxane units having a chromophore residue A of the formula IIa to those having a chromophore residue A of the formula IIb is not critical. Said ratio may be about 1:1 to about 19:1, preferably about 2:1 to about 9:1, more preferably about 4:1.

The polysiloxane compounds Ia or Ib wherein A is a reside of the formula IIa or IIb can be prepared as described in EP-B0538431 by silylation of the corresponding benzalmalonates according to the following reaction scheme:

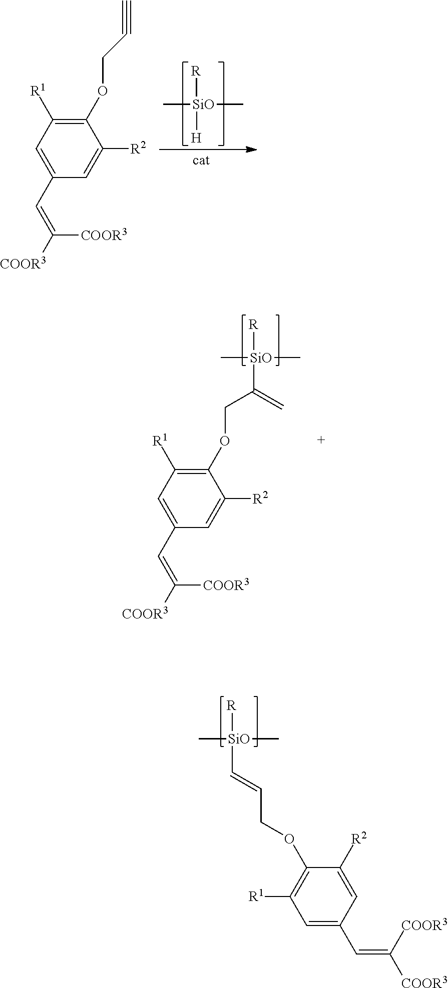

wherein R¹, R² and R³ are as defined above.

The silylation of the 4-(2-propinyloxy)phenyl methylene diethylester may be carried out employing known procedures for the addition of silicon bonded hydrogen atoms to groups containing aliphatic unsaturation. Such reactions are generally catalyzed by a platinum group metal or a complex of such a metal. Examples of catalysts which may be employed are platinum on carbon, chloroplatinic acid, platinum acetyl acetonate, complexes of platinum compounds with unsaturated compounds e.g. olefins and divinyl disiloxanes, complexes of rhodium and palladium compounds and complexes of platinum compounds supported on inorganic substrates. The addition reaction may be performed at reduced, atmospheric or increased pressure. A solvent can be used, e.g. toluene or xylene, in the reaction mixture although the presence of the solvent is not essential. It is also preferred to carry out the reaction at elevated reaction temperatures e.g. from about 50° C. to about 150° C.

Particularly preferred are compounds of the general formula Ia, wherein

X signifies methyl,

A signifies the group of the formula IIa or IIb,

R signifies methyl,

R¹ and R² signify hydrogen, methoxy or ethoxy or one or R¹ and R² is hydrogen and the other is methyl, methoxy or ethoxy,

R³ signifies methyl or ethyl,

R⁴ signifies hydrogen or methyl,

R⁵ and R⁶ signify hydrogen,

r is about 5 to 150,

s is about 2 to about 10, and

n has a value of 1.

Most preferred are linear polysiloxanes of the general formula Ia, wherein

X signifies methyl,

A signifies a group of the formula IIa or IIb,

R signifies methyl,

R¹ and R² signify hydrogen,

R³ signifies ethyl,

R⁴ signifies hydrogen,

R⁵ and R⁶ signify hydrogen,

r is a statistical mean of about 60,

s is a statistical mean of about 4, and

n has a value of 1.

These most preferred polysiloxane-based UV filter in all embodiments of the present invention is polysilicone-15 (INCI) which is commercially available under the tradename PARSOL® SLX at DSM Nutritional Products Ltd.

In all embodiments of the present invention, the amount of the polysiloxane-based UV filter, preferably having a chromophore residue of the benzalmalonate type, such as most preferably polysilicone-15, in the compositions according to the present invention is preferably selected in the range 0.1 to 15 wt.-%, such as in the range of 0.5 to 10 wt.-%, such as most preferably in the range of 1 to 5 wt.-% based on the total weight of the composition. Further ranges would be 0.2-13 wt-%, 0.3-12 wt-% or 0.4-11 wt %.

In all embodiments of the present invention, the amount of panthenol used in the topical compositions according to the present invention is advantageously selected in the range of 0.001 to 10 wt.-%, preferably in the range of 0.01 to 7 wt-%, more preferably in the range of 0.1 to 6 wt-% and most preferably in the range of 0.5 to 5 wt-% based on the total weight of the composition. Further preferred ranges are 0.5 to 4.5 wt-%, 0.6 to 4 wt-%, 0.6 to 3.5 wt-% panthenol based on the total weight of the composition.

In all embodiments of the present invention, the amount of the hydroxyacetophenone, such as preferably p-hydroxyacetophenone used in the topical compositions according to the present invention is advantageously selected in the range of 0.001 to 5 wt.-%, preferably in the range of 0.01 to 4 wt-%, more preferably in the range of 0.1 to 3 wt-% based on the total weight of the composition. Further preferred ranges are 0.005 to 4.5 wt-%, 0.05 to 4.3 wt-%, and 0.25 to 2.9 wt-% based on the total weight of the composition.

In one embodiment, the present invention relates to the topical composition according to the embodiments described herein for the use as sunscreen, respectively to the use of the topical composition according to the embodiments described herein as sunscreen.

In another embodiment, the invention relates to the use of panthenol, preferably in combination with a polysiloxane-based UV filter and a hydroxyacetophenone with all the definitions and preferences as given herein for reducing the stickiness and/or sand adherence on skin.

A further embodiment of the present invention relates to a method for reducing the stickiness of skin and/or the sand adherence onto skin to which a topical composition according to the present invention has been applied, said method encompassing the step of incorporating panthenol, preferably in combination with a polysiloxane-based UV filter and a hydroxyacetophenone with all the definitions and preferences as given herein into said topical composition and optionally appreciating the effect. Preferably, panthenol is used in an amount selected in the range of 0.001 to 10 wt.-%, preferably in the range of 0.01 to 7 wt-%, more preferably in the range of 0.1 to 6 wt-% and most preferably in the range of 0.5 to 5 wt-% based on the total weight of the composition.

In particular, one embodiment of the present invention relates to a method for the use of a topical composition comprising a polysiloxane-based UV filter, a hydroxyacetophenone and panthenol with all the definitions and preferences as given herein for reducing the stickiness and/or sand adherence on skin after application of said topical composition.

Besides the polysiloxane-based UV filter also further UV filters may be incorporated into the topical composition according to the present invention. These UV filters are all commercially available UV-filter substances such as (INCI names) methylene bis-benzotriazolyl tetramethylbutylphenol or tris-biphenyl triazine, phenylbenzimidazol sulfonic acid, 3-benzylidene camphor, octocrylene, ethylhexyl methoxycinnamate, ethyl hexylsalicylate, homosalate, ethylhexyl triazone, zinc oxide, bis-ethylhexyloxyphenol methoxyphenyl triazine, diethylhexyl butamido triazone, benzophenon-3, titanium dioxide, butyl methoxydibenzoylmethane, disodium phenyl dibenzimidazole tetrasulfonate and diethylamino hydroxybenzoyl hexyl benzoate without being limited thereto.

In a particular preferred embodiment, the topical compositions according to the present invention comprise, next to the polysiloxane-based UV filter, the hydroxyacetophenone and panthenol at least one additional UV-filter selected from the group of octocrylene, ethyl hexylsalicylate and butylmethoxydibenzoyl methane as well as mixtures thereof.

As the compositions according to the invention are intended for topical application, they comprise a physiologically acceptable medium, that is to say a medium compatible with keratinous substances, such as the skin, mucous membranes, and keratinous fibers. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment, the topical compositions according to the invention are light-protective preparations (sun care products), such as sun protection milks, sun protection lotions, sun protection creams, sun protection oils, sun blocks or tropical's or day care creams with a SPF (sun protection factor). Of particular interest are sun protection creams, sun protection lotions, sun protection milks and sun protection preparations.

The topical compositions according to the present invention may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

The topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples.

In one advantageous embodiment, O/W emulsifier is a phosphate ester emulsifier. The term phosphate ester emulsifier refers to phosphate esters emulsifier of formula (II)

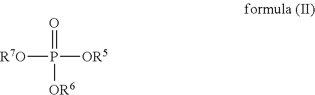

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C₆₋₁₀ Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular phosphate ester emulsifier according to the invention is potassium cetyl phosphate e.g. commercially available as Amphisol® K at DSM Nutritional Products Ltd Kaiseraugst.

Further suitable O/W emuslifiers according to the present invention encompass PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self-emulsifying), stearic acid, salts of stearic acid, polyglyceryl-3-methylglycosedistearate. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

Another particular suitable class of O/W emulsifiers are non-ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

Further suitable are commercially available polymeric emulsifiers such as hydrophobically modified polyacrylic acid such as Acrylates/C10-30 Alkyl Acrylate Crosspolymers which are commercially available under the tradename Pemulen® TR-1 and TR-2 by Noveon.

Another class of particularly suitable emulsifiers are polyglycerol esters or diesters of fatty acids also called polyglyceryl ester/diester (i.e. a polymer in which fatty acid(s) is/are bound by esterification with polyglycerine), such as e.g. commercially available at Evonik as Isolan GPS [INCI Name Polyglyceryl-4 Diisostearate/Polyhydroxystearate/Sebacate (i.e. diester of a mixture of isostearic, polyhydroxystearic and sebacic acids with Polyglycerin-4)] or Dehymuls PGPH available at Cognis (INCI Polyglyceryl-2 Dipolyhydroxystearate).

Also suitable are polyalkylenglycolether such as Brij 72 (Polyoxyethylen(2)stearylether) or Brij 721 (Polyoxyethylene (21) Stearyl Ether e.g. available at Croda.

The at least one O/W respectively Si/W emulsifier is preferably used in an amount of 0.5 to 10 wt.-% such as in particular in the range of 0.5 to 5 wt.-% such as most in particular in the range of 0.5 to 4 wt.-% based on the total weight of the composition.

Suitable W/O- or W/Si-emulsifiers according to the present invention are polyglyceryl-2-dipolyhydroxystearat, PEG-30 dipolyhydroxystearat, cetyl dimethicone copolyol, polyglyceryl-3 diisostearate polyglycerol esters of oleic/isostearic acid, polyglyceryl-6 hexaricinolate, polyglyceryl-4-oleate, polygylceryl-4 oleate/PEG-8 propylene glycol cocoate, magnesium stearate, sodium stearate, potassium laurate, potassium ricinoleate, sodium cocoate, sodium tallowate, potassium castorate, sodium oleate, and mixtures thereof. Further suitable W/Si-emulsifiers are Lauryl Polyglyceryl-3 Polydimethylsiloxyethyl Dimethicone and/or PEG-9 Polydimethylsiloxyethyl Dimethicone and/or Cetyl PEG/PPG-10/1 Dimethicone and/or PEG-12 Dimethicone Crosspolymer and/or PEG/PPG-18/18 Dimethicone. The at least one W/O emulsifier is preferably used in an amount of about 0.001 to 10 wt.-%, more preferably in an amount of 0.2 to 7 wt.-% with respect to the total weigh of the composition.

The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 7 wt.-%, such as most in particular in the range of 1 to 5 wt.-%, based on the total weight of the composition. Particular suitable co-surfactants are selected from the list of alkyl alcohols such as cetyl alcohol (Lorol C16, Lanette 16), cetearyl alcohol (Lanette 0), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof

The compositions in form of O/W emulsions according to the invention can be provided, for example, in all the formulation forms for O/W emulsions, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

Finally, a subject-matter of the invention is a method for the cosmetic treatment of keratinous substances such as in particular the skin, wherein a composition as defined above is applied to the said keratinous substances such as in particular to the skin. The method is in particular suitable to protect the skin against the adverse effects of UV-radiation such as in particular sun-burn and/or photoageing.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992), The Cosmetic, Toiletry and Fragrance Association, Inc. without being limited thereto.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH according to standard methods in the art.

The topical compositions according to the invention may further contain one or more emollients which soothe and soften the skin. As an example, the emollient may be dicaprylyl carbonate or C_{12_15} alkyl benzoate. Further emollients are silicone (dimethicone, cyclomethicone), vegetable oils (grape seed, sesame seed, jojoba, etc.), butters (cocoa butter, shea butter), alcohols (stearyl alcohol, cetyl alcohol), and petrolatum derivatives (petroleum jelly, mineral oil).

Moreover, the topical compositions according to the present invention advantageously comprise further preservatives or preservative booster. Preferably, the additional preservatives respectively preservative booster is selected from the group consisting of phenoxyethanol, ethylhexylglycerin, glyceryl caprylate, caprylyl glycol, 1,2-hexanediol, propanediol, propylene glycol as well as mixtures thereof. When present, the preservative respectively preservative booster is preferably used in an amount of 0.01 to 2 wt.-%, more preferably in an amount of 0.05 to 1.5 wt.-%, most preferably in an amount of 0.1 to 1.0 wt.-%, based on the total weight of the composition. It is particularly preferred, that the cosmetic compositions according to the invention does not contain any further/other preservatives such as e.g. parabens and/or methylisothiazolidine.

The following examples are provided to further illustrate the compositions and effects of the present invention. These examples are illustrative only and are not intended to limit the scope of the invention in any way.

### EXAMPLES

The formulations (O/W emulsions) as outlined in table 1 have been prepared according to standard methods in the art. Then the sand repellence has been tested according to the method as outlined below:
Apply 30 mg of cream on PMMA plate and distribute homogenously
Let it dry for 15 minutes
Weigh the PMMA plate
Put 30 g of sand on top of the PMMA plate such as that the surface is completely covered
Let it dry for 15 minutes
Turn the PMMA plate 180° without shaking
Weigh the PMMA plate
Calculate the amount of sand sticking on the plate
Repeat test with 4 plates per sample

The results are outlined in Table 1

TABLE 1

| INCI | Ref 1 | Ref 2 | Inv 1 | Inv 2 |
|---|---|---|---|---|
| Butyl Methoxydibenzoyl Methane | 4.00 | 4.00 | 4.00 | 4.00 |
| Octocrylene | 8.00 | 8.00 | 8.00 | 8.00 |
| Ethylhexyl Salicylate | 5.00 | 5.00 | 5.00 | 5.00 |
| Potassium Cetyl Phosphate | 1.50 | 1.50 | 1.50 | 1.50 |
| Cetyl alcohol | 3.00 | 3.00 | 3.00 | 3.00 |
| Dicaprylyl Carbonate | 8.00 | 8.00 | 8.00 | 8.00 |
| C12-15 Alkyl Benzoate | 8.00 | 8.00 | 8.00 | 8.00 |
| Aqua | Ad 100 | Ad 100 | Ad 100 | Ad 100 |
| Glycerin | 3.00 | 3.00 | 3.00 | 3.00 |
| Phenoxyethanol, Ethylhexylglycerin | 1.00 | 1.00 | 1.00 | 1.00 |
| Xanthan Gum | 0.30 | 0.30 | 0.30 | 0.30 |
| Hydroxyacetophenone | 0.50 | 0.50 | 0.50 | 0.50 |
| Polysilicone 15 | 3.00 | — | 3.00 | 3.00 |
| Panthenol | | 1.00 | 1.00 | 3.00 |
| Remaining amount of sand on the plate | 1.10 | 0.98 | 0.88 | 0.87 |
| % reduction (less sand) vs. Ref 1 | | | −20% | −21% |
| % reduction (less sand) vs. Ref 2 | | | −10% | −11% |

As can be retrieved from table 1, the combination according to the present invention results in a synergistic reduction of sand sticking to the plates, i.e. a synergistically improved sand repellence.

1. A topical composition comprising a polysiloxane-based UV filter, a hydroxyacetophenone and panthenol.

2. The topical composition according to claim 1, wherein the polysiloxane-based UV filter has a chromophore residue of the benzalmalonate type.

3. The topical composition according to claim 1, wherein the polysiloxane-based UV filter is a compound according to formula Ia or Ib: 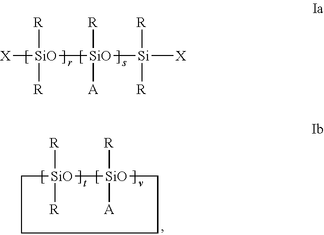
wherein
X is R or A;
A is selected from formula IIa, IIb or IIc: 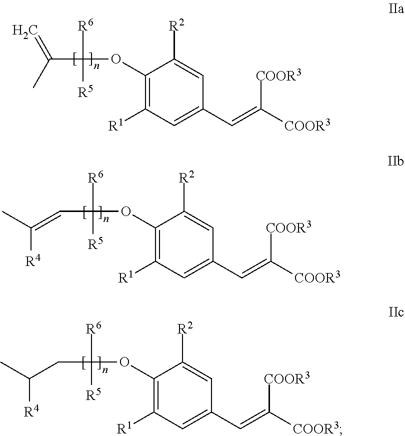
R is hydrogen, C₁₋₆-alkyl or phenyl;
R¹ and R² are each independently hydrogen, hydroxy, C₁₋₆-alkyl or C₁₋₆-alkoxy;
R³ is C₁₋₆-alkyl;
R⁴ is hydrogen or C₁₋₆-alkyl;
R⁵ and R⁶ are each independently hydrogen or C₁₋₆-alkyl;
r is from 0 to 250;
s is from 0 to 20;
r+s is at least 3;
t is from 0 to 10;
v is from 0 to 10;
v+t is at least 3; and
n is from 1 to 6;
with the proviso that when s is 0, at least one X is A.

4. The topical composition according to claim 1, wherein the polysiloxane-based UV filter is polysilicone-15.

5. The topical composition according to claim 1, wherein the hydroxyacetophenone is p-hydroxyacetophenone.

6. The topical composition according to claim 1, wherein the amount of the polysiloxane-based UV filter is selected in the range 0.1 to 15 wt.-%, preferably in the range of 0.5 to 10 wt.-%, most preferably in the range of 1 to 5 wt.-%, based on the total weight of the composition.

7. The topical composition according to claim 1, wherein panthenol is used in an amount selected in the range of 0.001 to 10 wt.-%, preferably in the range of 0.01 to 7 wt-%, more preferably in the range of 0.1 to 6 wt-% and most preferably in the range of 0.5 to 5 wt-% based on the total weight of the composition.

8. The topical composition according to claim 1, wherein hydroxyacetophenone is used in an amount selected in the range of 0.001 to 5 wt.-%, preferably in the range of 0.01 to 4 wt-%, more preferably in the range of 0.1 to 3 wt-% based on the total weight of the composition.

9. The topical composition according to claim 1 comprising at least one additional UV-filter selected from the group consisting of octocrylene, ethyl hexylsalicylate and butylmethoxydibenzoyl methane as well as mixtures thereof.

10. The topical composition according to claim 1, wherein the composition is in the form of an oil-in-water (O/W) emulsion comprising an oily phase dispersed in an aqueous phase in the presence of an O/W emulsifier.

11. The topical composition according to claim 1, wherein the O/W emulsifier is potassium cetyl phosphate.

12. Use of the topical composition according to claim 1 as sunscreen.

13. Use of panthenol, a hydroxyacetophenone and a polysiloxane-based UV filter as defined in claim 1 for reducing the stickiness of skin and/or the sand adherence onto skin.

14. Method for reducing the stickiness of skin and/or the sand adherence onto skin to which a topical composition has been applied, said method encompassing the step of incorporating panthenol, a hydroxyacetophenone and a polysiloxane-based UV filter as defined in claim 1 into said topical composition.

15. Method according to claim 14, wherein panthenol is used in an amount selected in the range of 0.001 to 10 wt.-%, preferably in the range of 0.01 to 7 wt-%, more preferably in the range of 0.1 to 6 wt-% and most preferably in the range of 0.5 to 5 wt-% based on the total weight of the composition.
